(19) United States
(12) Patent Application Publication
(10) Pub. No.: US 2024/0199544 A1
(43) Pub. Date: Jun. 20, 2024

(54) CRYSTALLINE FORMS OF N,N-DIMETHYLTRYPTAMINE AND METHODS OF USING THE SAME

(71) Applicant: Atai Therapeutics, Inc., Berlin (DE)

(72) Inventors: Majed Fawaz, Foxborough, MA (US); Setu Kasera, Stuttgart (DE)

(21) Appl. No.: 18/526,500

(22) Filed: Dec. 1, 2023

Related U.S. Application Data
(60) Provisional application No. 63/429,369, filed on Dec. 1, 2022.

(51) Int. Cl. C07D 209/16; A61K 31/4045

(52) U.S. Cl. CPC C07D 209/16; A61K 31/4045

(57) ABSTRACT

The present disclosure provides crystalline forms of N,N-dimethyltryptamine,

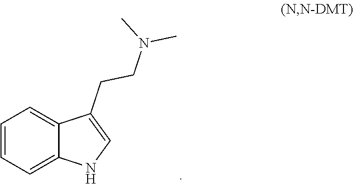

Also provided herein are methods of using crystalline forms of N,N-DMT and compositions comprising the same, e.g., in the treatment of mental health diseases and/or disorders.

### BACKGROUND OF THE INVENTION

N,N-dimethyltryptamine (N,N-DMT) has therapeutic value as a psychedelic, with intrinsic properties making it an attractive possible medication, especially for neurological diseases and conditions. Crystalline forms of active pharmaceutical ingredients offer the best means for controlling important physiochemical qualities, such as stability, solubility, bioavailability, particle size, bulk density, flow properties, polymorphic content, and other properties. Two crystalline forms of N,N-DMT have been previously identified and designated as “Form I” (Bergin et al. Acta Crystallogr., Sect. B24, 882 (1968) and Gaujac et al. Talanta 106, 394 (2013)) and “Form II” (Falkenberg et al. Acta Crystallogr., Sect B28, 3075 (1972)). There remains a need in the art to develop additional crystalline forms of N,N-DMT.

### BRIEF SUMMARY OF THE INVENTION

The present disclosure relates to crystalline forms of N,N-dimethyltryptamine

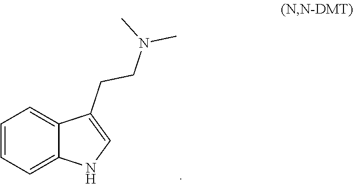

In embodiments, the present disclosure provides Form IV of N,N-dimethyltryptamine.

In embodiments, Form IV is characterized by peaks in an X-ray powder diffraction (XRPD) pattern at 13.6±0.2, 20.8±0.2, and 17.8±0.2 °2θ. In embodiments, Form IV is further characterized by at least one XRPD peak selected from 13.9±0.2, 14.0±0.2, 15.4±0.2, 18.6±0.2, 23.6±0.2, 24.5±0.2, 26.0±0.2, or 26.7±0.2 °2θ.

In embodiments, Form IV is characterized by peaks in an XRPD pattern at 13.6±0.2, 17.8±0.2, and 20.8±0.2 and at least one XRPD peak selected from 13.9±0.2, 14.0±0.2, 15.4±0.2, 18.6±0.2, 23.6±0.2, 24.5±0.2, 26.0±0.2, or 26.7=0.2 °2θ.

In embodiments, Form IV is characterized by peaks in an XRPD pattern at 13.6±0.2, 13.9±0.2, 14.0±0.2, 15.4±0.2, 17.8±0.2, 18.6±0.2, 20.8±0.2, 23.6±0.2, 24.5±0.2, 26.0±0.2, and 26.7±0.2 °2θ.

In embodiments, Form IV is characterized by peaks in an XRPD pattern at 7.7±0.2, 10.4±0.2, 11.9±0.2, 13.0±0.2, 13.6±0.2, 13.9±0.2, 14.0±0.2, 15.2±0.2, 15.4±0.2, 16.2±0.2, 16.9±0.2, 17.5±0.2, 17.8±0.2, 18.1±0.2, 18.6±0.2, 19.3±0.2, 19.8±0.2, 20.8±0.2, 21.1±0.2, 21.6±0.2, 22.4±0.2, 22.7±0.2, 23.6±0.2, 23.8±0.2, 24.5±0.2, 24.8±0.2, 25.2±0.2, 26.1±0.2, 26.7±0.2, 26.9±0.2, 27.5±0.2, 28.0±0.2, 28.2±0.2, 28.6±0.2, 28.8±0.2, 29.3±0.2, 29.5±0.2, 29.6±0.2, 30.2±0.2, 30.4±0.2, 30.6±0.2, 30.9±0.2, 31.1±0.2, 31.6±0.2, 31.9±0.2, 32.8±0.2, 33.2±0.2, 33.7±0.2, 34.5±0.2, and 35.3±0.2 °2θ.

In embodiments, Form IV is characterized an XRPD pattern substantially similar to that shown in FIG. 3.

In embodiments, Form IV exhibits a Differential Scanning Calorimetry (DSC) thermogram comprising an endotherm peak with an onset at 69±5 ° C. In embodiments, Form IV exhibits a DSC thermogram comprising an endothermic peak at 70±5 ° C.

In embodiments, Form IV exhibits substantially no weight loss at a temperature under 225±5° C.as measured by thermogravimetric (TGA) analysis.

In embodiments, Form IV exhibits a melting point of 69±5° C.

In embodiments, the present disclosure provides Form III of N,N-dimethyltryptamine.

In embodiments, Form III is characterized by peaks in an XRPD pattern at 7.6±0.2 and 15.2±0.2 In embodiments, Form III is further characterized by at least one XRPD peak selected from 19.2±0.2 ° 20, 19.6±0.2 or 23.0=0.2 °2θ.

In embodiments, Form III is characterized by peaks in an XRPD pattern at 7.6±0.2, 15.2±0.2, 19.2±0.2, 19.6±0.2, and 22.9±0.2 °2θ.

In embodiments, Form III is characterized by peaks in an XRPD pattern at 7.6±0.2, 15.2±0.2, 16.8±0.2, 19.2±0.2, 19.6±0.2, 20.0±0.2, 20.4±0.2, 20.7±0.2, 21.5±0.2, 22.4±0.2, 22.9±0.2, 23.1±0.2, 26.3±0.2, 27.1±0.2, 27.8±0.2, 28.5±0.2, 30.7±0.2, and 31.56=0.2 °2θ.

In embodiments, Form III is characterized an XRPD pattern substantially similar to that shown in FIG. 2.

In embodiments, Form III exhibits a DSC thermogram comprising an endotherm peak with an onset at 67±5° C. In embodiments, Form III exhibits a DSC thermogram comprising an endothermic peak at 68±5° C.

In embodiments, Form III exhibits substantially no weight loss at a temperature under 200±5° C. as measured by TGA analysis.

In embodiments, Form III exhibits a melting point of 39±5° C.

In embodiments, the present disclosure provides pharmaceutical compositions comprising Form IV of N,N-dimethyltryptamine. In embodiments, the pharmaceutical composition further comprises a pharmaceutically acceptable excipient. In embodiments, the present disclosure provides an oral dosage form comprising Form IV of N,N-dimethyltryptamine.

In embodiments, the present disclosure provides pharmaceutical compositions comprising Form III of N,N-dimethyltryptamine. In embodiments, the pharmaceutical composition further comprises a pharmaceutically acceptable excipient. In embodiments, the present disclosure provides an oral dosage form comprising Form III of N,N-dimethyltryptamine.

The present disclosure provide methods of treating post-traumatic stress disorder (PTSD) in a subject in need thereof, comprising administering a therapeutically effective amount of a Form IV of N,N-dimethyltryptamine, Form III of N,N-dimethyltryptamine, a pharmaceutical composition, or an oral dosage form described herein to the subject.

The present disclosure provide methods of treating post-traumatic stress disorder (PTSD) in a subject in need thereof, comprising administering a therapeutically effective amount of a Form IV of N,N-dimethyltryptamine, Form III of N,N-dimethyltryptamine, a pharmaceutical composition, or an oral dosage form described herein to the subject. In embodiments, the depression is a major depressive disorder (MDD) or treatment-resistant depression (TRD).

The present disclosure provide methods of treating an anxiety disorder in a subject in need thereof, comprising administering a therapeutically effective amount of a Form IV of N,N-dimethyltryptamine, Form III of N,N-dimethyltryptamine, a pharmaceutical composition, or an oral dosage form to the subject.

### BRIEF DESCRIPTION OF THE DRAWINGS

FIG. 1 shows the XRPD diffractogram of a sample of Form I of N,N-DMT.

FIG. 2 shows the XRPD diffractogram of a sample of Form III of N,N-DMT.

FIG. 3 shows the XRPD diffractogram of a sample of Form IV of N, N-DMT.

FIG. 4 shows an atomic displacement ellipsoid diagram of Form I of N,N-DMT.

FIG. 5 shows an atomic displacement ellipsoid diagram of Form IV of N,N-DMT.

FIG. 6 shows the XRPD diffractogram of a sample of the Form I of N,N-DMT before (top plot) and after (bottom plot) dynamic vapor sorption (DVS).

FIGS. 7 and 8 show DCS thermograms of a sample of Form I of N,N-DMT.

FIG. 9 shows a DSC thermogram (top) collected at a heating rate of 10° C/minute and a TGA thermogram (bottom) of a sample of Form I of N,N-DMT.

FIG. 10 shows a DSC thermogram collected at a heating rate of 2° C./minute of a sample of Form I of N,N-DMT.

FIG. 11 shows a DSC thermogram (top) collected at a heating rate of 10° C./minute and a TGA thermogram (bottom) of a sample of Form III of N,N-DMT.

FIG. 12 shows a DSC thermogram collected at a heating rate of 2° C./minute of a sample of Form III of N,N-DMT.

FIG. 13 shows a DSC thermogram (top) collected at a heating rate of 10° C./minute and a TGA thermogram (bottom) of a sample of Form IV of N,N-DMT.

FIG. 14 shows a DSC thermogram (top) collected at a heating rate of 10° C./minute and a TGA thermogram (bottom) of a sample of Form IV of N,N-DMT.

FIG. 15 shows a DSC thermogram collected at a heating rate of 2° C./minute of a sample of Form IV of N,N-DMT.

FIG. 16 shows polarized light photomicrographs of a sample of Form I of N,N-DMT.

FIG. 17 shows polarized light photomicrographs of a sample of Form IV of N,N-DMT.

FIG. 18 shows an in situ cycling DSC thermogram of amorphous N,N-DMT starting from a sample of Form IV.

FIG. 19 shows a 1H NMR spectrum for N,N-DMT.

### DETAILED DESCRIPTION OF THE INVENTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

The terms “administer,” “administering” or “administration” as used herein refer to administering a compound or pharmaceutically acceptable salt of the compound or a composition or formulation comprising the compound or pharmaceutically acceptable salt of the compound to a patient.

The term “preventing” as used herein with regard to a patient or subject, refers to preventing the onset of disease development if none had occurred, preventing the disease or disorder from occurring in a subject or a patient that may be predisposed to the disorder or disease but has not yet been diagnosed as having the disorder or disease, and/or preventing further disease/disorder development if already present.

The term “substantially similar” as used herein with regards to an analytical spectrum, such as an XRPD pattern, means that a spectrum resembles the reference spectrum in both the peak locations and their relative intensities, allowing for variability appropriate in the art. For example, two spectra may be regarded as “substantially similar” when the two spectra share defining characteristics sufficient to differentiate them from a spectrum obtained for a different solid form. In embodiments, spectra or characterization data that are substantially similar to those of a reference crystalline form are understood by those of ordinary skill in the art to correspond to the same crystalline form as the particular reference. In analyzing whether spectra or characterization data are substantially similar, a person of ordinary skill in the art understands that particular characterization data points may vary to a reasonable extent while still describing a given solid form, due to, for example, experimental error and routine sample-to-sample analysis.

The term “treating” as used herein with regard to a patient or subject, refers to improving at least one symptom of the patient's or subject's disorder. In some embodiments, treating can be improving, or at least partially ameliorating a disorder or one or more symptoms of a disorder.

The term “therapeutically effective” applied to dose or amount refers to that quantity of a compound or pharmaceutical formulation that is sufficient to result in a desired clinical benefit after administration to a patient or subject in need thereof.

All XRPD peaks and patterns are given in °2θ using Cu Kα1 radiation at a wavelength of 1.5406 Å. The values of degree 2θ allow appropriate error margins. For example, the degree 2θ of about “17.48±0.2” denotes a range from about 17.46 to 17.50 degree 2θ. Depending on the sample preparation techniques, the calibration techniques applied to the instruments, human operational variation, and etc., those skilled in the art recognize that the appropriate error of margins for a XRPD can be ±0.2, which includes any value below ±0.2 such as ±0.1; ±0.05; or less.

TGA and DSC thermograms for a given crystalline form of the same compound will vary within a margin of error. The values of a single peak, expressed in degree Celsius, allow appropriate error margins. Typically, the error margins are represented by “±”. For example, the single peak characteristic value of about “120±5” denotes a range from about 115 to 125. Depending on the sample preparation techniques, the calibration techniques applied to the instruments, human operational variations, and etc., those skilled in the art recognize that the appropriate error of margins for a single peak characteristic value can be ±5, which includes any value below ±5 such as ±4, ±3.5, ±3, ±2.5; ±2.0; ±1.5; ±1.0; ±0.5; or less.

The following description includes information that may be useful in understanding the present invention. It is not an admission that any of the information provided herein is prior art or relevant to the presently claimed inventions, or that any publication specifically or implicitly referenced is prior art.

### Crystalline Forms of N,N-dimethyltryptamine (N,N-DMT)

In one aspect, the present disclosure provides crystalline forms of N,N-DMT. In embodiments, the crystalline form of N,N-DMT is Form III. In embodiments, the crystalline form of N,N-DMT is Form IV. In embodiments, the crystalline form of N,N-DMT is a mixture of Form III and Form IV.

In embodiments, the crystalline form of N,N-DMT comprises a mixture of one or more forms of N,N-DMT (e.g., Form III or IV). In some embodiments, the crystalline form of N,N-DMT comprises a substantially pure form of one form of N,N-DMT. In embodiments, the substantially pure form is Form III. In embodiments, the substantially pure form is Form IV.

In embodiments, the crystalline form of N,N-DMT comprises greater than about 99.9%, about 99.8%, about 99.7%, about 99.6%, about 99.5%, about 99.4%, about 99.3%, about 99.2%, about 99.1%, or about 99.0% of one form of N,N-DMT (e.g., Form III and/or Form IV).

In embodiments, the crystalline form of N,N-DMT comprises greater than about 99%, 98%, 97%, 96%, 95%, 94%, 93%, 92%, 91%, or 90% of one form of N,N-DMT. In some embodiments, the crystalline form of N,N-DMT comprises greater than about 90%, 85%, 80%, 75%, 70%, 65%, 60%, 55%, 50%, 45%, or 40% of one form of N,N-DMT (e.g., Form III and/or Form IV).

I. Form III

In embodiments, the crystalline form of N,N-DMT is Form III.

In embodiments, Form III is characterized by peaks in a XRPD pattern at 7.6±0.2 and 15.2±0.2. In embodiments, the variance at any of these peaks is ±0.1 °2θ.

In embodiments, Form III is characterized by peaks in a XRPD pattern at 7.6±0.2 and 15.2±0.2 and least one peak in an XRPD pattern selected from 19.2±0.2 °2θ, 19.6±0.2 or 23.0±0.2 °2θ. In embodiments, the variance at any of these peaks is ±0.1 °2θ.

In embodiments, Form III is characterized by peaks in a XRPD pattern at 7.6±0.2, 15.2±0.2, 19.2±0.2, and 19.6±0.2 °2θ. In embodiments, the variance at any of these peaks is ±0.1 °2θ.

In embodiments, Form III is characterized by peaks in a XRPD pattern at 7.6±0.2, 15.2±0.2, 19.2±0.2, and 23.0±0.2 °2θ. In embodiments, the variance at any of these peaks is ±0.1 °2θ.

In embodiments, Form III is characterized by peaks in an XRPD pattern at 7.6±0.2, 15.2±0.2, 19.2±0.2, 19.6±0.2, and 22.9±0.2 °2θ. In embodiments, the variance at any of these peaks is ±0.1 °2θ.

In embodiments, Form III is characterized by peaks in an XRPD pattern at 7.6±0.2, 15.2±0.2, 16.8±0.2, 19.2±0.2, 19.6±0.2, 20.0±0.2, 20.4±0.2, 20.7±0.2, 21.5±0.2, 22.4±0.2, 22.9±0.2, 23.1±0.2, 26.3±0.2, 27.1±0.2, 27.8±0.2, 28.5±0.2, 30.7±0.2, and 31.56=0.2 °2θ.

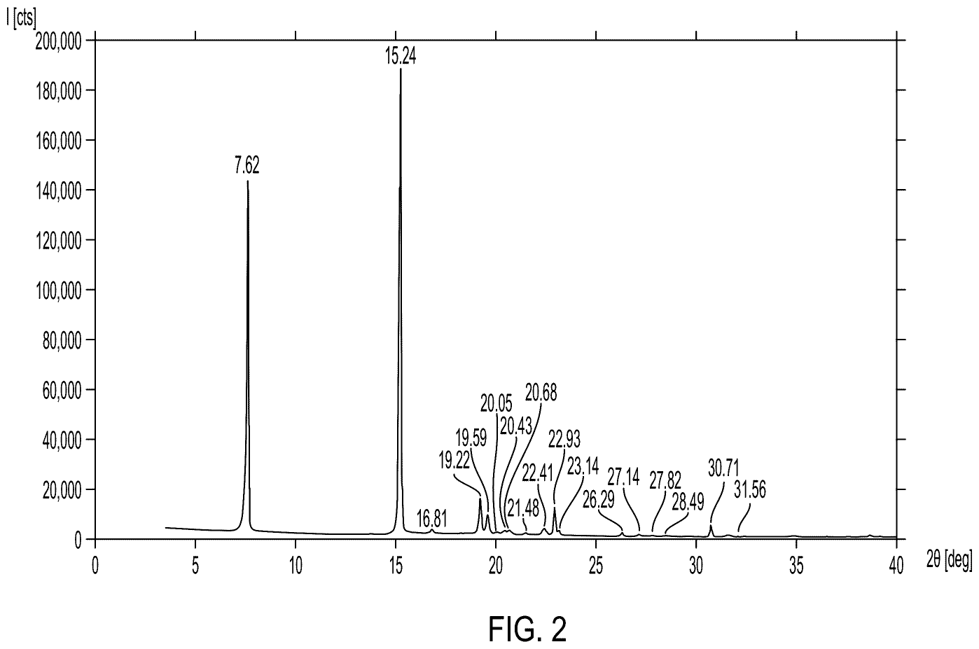

In embodiments, Form III is characterized by an XRPD pattern substantially similar to that shown in FIG. 2.

In embodiments, Form III is characterized by an XRPD pattern comprising peaks show in Table 4.

In embodiments, Form III is characterized by an XRPD pattern comprising peaks show in Table 5.

In embodiments, Form III exhibits a DSC thermogram comprising an endotherm peak with an onset at 67±5° C. In embodiments, Form III exhibits a DSC thermogram comprising an endothermic peak at 68±5° C.

In embodiments, Form III exhibits substantially no weight loss at a temperature under 200±5° C.as measured by TGA analysis.

In embodiments, Form III exhibits a melting point of 39±5° C.

II. Form IV

In embodiments, the crystalline form of N,N-DMT is Form IV.

In embodiments, Form IV is characterized by peaks in a XRPD pattern at 13.6±0.2, 20.8±0.2, and 17.8±0.2 ° 20. In embodiments, the variance at any of these peaks is ±0.1 ° 20.

In embodiments, Form IV is characterized by peaks in a XRPD pattern at 13.6±0.2, 20.8±0.2, and 17.8±0.2 °2θ and least one peak in an XRPD pattern selected from 13.9±0.2, 14.0±0.2, 15.4±0.2, 18.6±0.2, 23.6±0.2, 24.5±0.2, 26.0±0.2, or 26.7±0.2 ° 20. In embodiments, the variance at any of these peaks is ±0.1 °2θ.

In embodiments, Form IV is characterized by peaks in a XRPD pattern at 13.6±0.2, 13.9±0.2, 17.8±0.2, and 20.8±0.2 °2θ. In embodiments, the variance at any of these peaks is ±0.1 °2θ.

In embodiments, Form IV is characterized by peaks in a XRPD pattern at 13.6±0.2, 15.4±0.2, 17.8±0.2, and 20.8±0.2 °2θ. In embodiments, the variance at any of these peaks is ±0.1 °2θ.

In embodiments, Form IV is characterized by peaks in a XRPD pattern at 13.6±0.2, 17.8±0.2, 18.6±0.2, and 20.8±0.2 °2θ. In embodiments, the variance at any of these peaks is ±0.1 °2θ.

In embodiments, Form IV is characterized by peaks in a XRPD pattern at 13.6±0.2, 17.8±0.2, 20.8±0.2, and 23.6±0.2 °2θ. In embodiments, the variance at any of these peaks is ±0.1 °2θ.

In embodiments, Form IV is characterized by peaks in a XRPD pattern at 13.6±0.2, 17.8±0.2, 20.8±0.2, and 24.5±0.2 °2θ. In embodiments, the variance at any of these peaks is ±0.1 °2θ.

In embodiments, Form IV is characterized by peaks in a XRPD pattern at 13.6±0.2, 17.8±0.2, 20.8±0.2, and 26.0±0.2 °2θ. In embodiments, the variance at any of these peaks is ±0.1 °2θ.

In embodiments, Form IV is characterized by peaks in a XRPD pattern at 13.6±0.2, 17.8±0.2, 20.8±0.2, and 26.7±0.2 °2θ. In embodiments, the variance at any of these peaks is ±0.1 °2θ.

In embodiments, Form IV is characterized by peaks in a XRPD pattern at 13.6±0.2, 20.8±0.2, and 17.8±0.2, and at least one XRPD peak selected from 13.9±0.2, 14.0±0.2, 15.4±0.2, 18.6±0.2, 23.6±0.2, 24.5±0.2, 26.0±0.2, or 26.7±0.2 °2θ. In embodiments, the variance at any of these peaks is ±0.1 °2θ.

In embodiments, Form IV is characterized by peaks in a XRPD pattern at 13.6±0.2, 13.9±0.2, 14.0±0.2, 15.4±0.2, 17.8±0.2, 18.6±0.2, 20.8±0.2, 23.6±0.2, 24.5±0.2, 26.0±0.2, and 26.7±0.2 °2θ. In embodiments, the variance at any of these peaks is ±0.1 °2θ.

In embodiments, Form IV is characterized by peaks in an XRPD pattern at 7.7±0.2, 10.4±0.2, 11.9±0.2, 13.0±0.2, 13.6±0.2, 13.9±0.2, 14.0±0.2, 15.2±0.2, 15.4±0.2, 16.2±0.2, 16.9±0.2, 17.5±0.2, 17.8±0.2, 18.0±0.2, 18.6±0.2, 19.3±0.2, 19.8±0.2, 20.8±0.2, 21.1±0.2, 21.6±0.2, 22.4±0.2, 22.7±0.2, 23.6±0.2, 23.8±0.2, 24.5±0.2, 24.8±0.2, 25.2±0.2, 26.1±0.2, 26.7±0.2, 26.9±0.2, 27.5±0.2, 28.0±0.2, 28.2±0.2, 28.6±0.2, 28.8±0.2, 29.3±0.2, 29.5±0.2, 29.6±0.2, 30.2±0.2, 30.4±0.2, 30.6±0.2, 30.9±0.2, 31.1±0.2, 31.6±0.2, 31.9±0.2, 32.8±0.2, 33.2±0.2, 33.7±0.2, 34.5±0.2, and 35.3±0.2 °2θ. In embodiments, the variance at any of these peaks is ±0.1 °2θ.

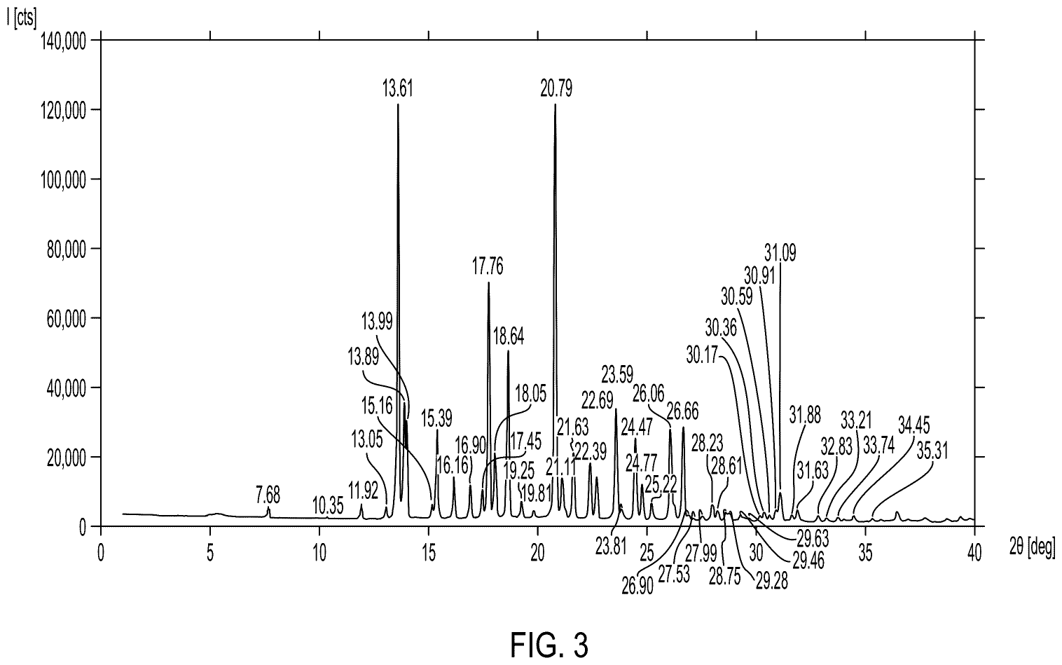

In embodiments, Form IV is characterized an XRPD pattern substantially similar to that shown in FIG. 3.

In embodiments, Form IV is characterized by an XRPD pattern comprising peaks show in Table 6.

In embodiments, Form IV is characterized by an XRPD pattern comprising peaks show in Table 7.

In embodiments, Form IV exhibits a DSC thermogram comprising an endotherm peak with an onset at 69±5° C. In embodiments, Form IV exhibits a DSC thermogram comprising an endothermic peak at 70±5° C.

In embodiments, Form IV exhibits substantially no weight loss at a temperature under 225±5° C.as measured by TGA analysis.

In embodiments, Form IV exhibits a melting point of 69±5° C.

### Pharmaceutical Compositions

In one aspect, the present disclosure provides compositions comprising a at least one crystalline form of N,N-DMT described herein (e.g., Form III and/or Form IV) and one or more excipients. In embodiments, the composition is a pharmaceutical composition comprising a crystalline form of N,N-DMT, and one or more pharmaceutically acceptable excipients.

In embodiments, the pharmaceutical composition comprises Form III of N,N-DMT.

In embodiments, the pharmaceutical composition comprises Form IV of N,N-DMT.

In embodiments, the composition comprises a pharmaceutically acceptable carrier. In embodiments, a pharmaceutically acceptable carrier includes a pharmaceutically acceptable excipient, binder, adjuvant, and/or diluent. In embodiments, suitable pharmaceutically acceptable carriers include, but are not limited to, inert solid fillers or diluents and sterile aqueous or organic solutions. In embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21th Edition (Lippincott Williams & Wilkins, 2005).

In embodiments, one or more crystalline forms of N,N-DMT (e.g., Form III and/or Form IV) of the present disclosure are formulated for administration by a variety of means including orally, parenterally, by inhalation spray, topically, or rectally in formulations containing pharmaceutically acceptable carriers, adjuvants, vehicles, or mixtures thereof. The term parenteral as used here includes subcutaneous, intravenous, intramuscular, and intraarterial injections with a variety of infusion techniques. Intraarterial and intravenous injection as used herein includes administration through catheters.

In embodiments, the present disclosure provides oral dosage forms comprising a composition described herein.

In embodiments, the oral dosage form is a solid dosage form such as a tablet, capsule, pill, powder, or granule. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art.

### Therapeutic Methods

In one aspect, the present disclosure provides methods of treating or preventing neurological disorders in a subject in need thereof, the methods comprising administering a therapeutically effective amount of a crystalline form of N,N-DMT disclosed herein (e.g., Form III and/or Form IV) or a pharmaceutical composition thereof to the subject.

In embodiments, the neurological disorder is a mood disorder. In embodiments, the mood disorder is clinical depression, postnatal depression or postpartum depression, perinatal depression, atypical depression, melancholic depression, psychotic major depression, cationic depression, seasonal affective disorder, dysthymia, double depression, depressive personality disorder, recurrent brief depression, major depressive disorder, minor depressive disorder, bipolar disorder or manic depressive disorder, depression caused by chronic medical conditions, treatment-resistant depression, refractory depression, suicidality, suicidal ideation, or suicidal behavior. In embodiments, the method described herein provides therapeutic effect to a subject suffering from depression (e.g., moderate or severe depression). In embodiments, the mood disorder is associated with neuroendocrine diseases and disorders, neurodegenerative diseases and disorders (e.g., epilepsy), movement disorders, tremor (e.g., Parkinson's Disease), or women's health disorders or conditions. In embodiments, the mood disorder is depression. In embodiments, the mood disorder is treatment-resistant depression or major depressive disorder. In embodiments, the mood disorder is major depressive disorder. In embodiments, the mood disorder is treatment-resistant depression.

In embodiments, the present disclosure provides methods of treating or preventing PTSD, mood disorders, general anxiety disorder, addictive disorders, and/or drug dependence in a subject in need thereof, the methods comprising administering a therapeutically effective amount of a crystalline form of N,N-DMT disclosed herein (e.g., Form III and/or Form IV) or a pharmaceutical composition thereof to the subject.

In embodiments, the present disclosure provides methods of treating or preventing PTSD in a subject in need thereof, the methods comprising administering a therapeutically effective amount of a crystalline form of N,N-DMT disclosed herein (e.g., Form III and/or Form IV) or a pharmaceutical composition thereof to the subject.

In embodiments, the present disclosure provides methods of treating or preventing behavioral or mood disorders in a subject in need thereof, the methods comprising administering a therapeutically effective amount of a crystalline form of N,N-DMT disclosed herein (e.g., Form III and/or Form IV) or a pharmaceutical composition thereof to the subject. In embodiments, the behavioral or mood disorder includes anxiety, such as social anxiety in autistic subjects (e.g., autistic adults) and anxiety related to life-threatening illnesses. In embodiments, the behavioral or mood disorder includes stress (where moderation thereof is measured, e.g., by effects on amygdala responses). In embodiments, the anxiety disorder is panic disorder, obsessive-compulsive disorder, and/or general anxiety disorder. In embodiments, the subject suffers from a lack of motivation, attention, lack of accuracy in memory recall, speed of response, perseveration, and/or cognitive engagement. Further examples include depression (e.g., MDD or TRD), attention disorders, disorders of executive function and/or cognitive engagement, obsessive compulsive disorder, bipolar disorder, panic disorder, phobia, schizophrenia, psychopathy, antisocial personality disorder and/or neurocognitive disorders.

In embodiments, the present disclosure provides methods for treating or preventing an addictive disorder in a subject in need thereof, the methods comprising administering a therapeutically effective amount of a crystalline form of N,N-DMT disclosed herein (e.g., Form III and/or Form IV) or a pharmaceutical composition thereof to the subject. In embodiments, the addictive disorder is alcohol abuse, substance abuse, smoking, obesity, or mixtures thereof. In embodiments, the disorder is an cating disorder (e.g., anorexia nervosa, bulimia, nervosa, binge eating disorder, etc.) or an auditory disorder.

In embodiments, the present disclosure provides methods for treating or preventing an impulsive disorder in a subject in need thereof, the methods comprising administering a therapeutically effective amount of a crystalline form of N,N-DMT disclosed herein (e.g., Form III and/or Form IV) or a pharmaceutical composition thereof to the subject. In embodiments, the impulsive disorder is attention deficit hyperactivity disorder (ADHD), attention deficit disorder (ADD), Tourette's syndrome, autism, or combinations thereof.

In embodiments, the present disclosure provides methods for treating or preventing a compulsive disorder in a subject in need thereof, the methods comprising administering a therapeutically effective amount of a crystalline form of N,N-DMT disclosed herein (e.g., Form III and/or Form IV) or a pharmaceutical composition thereof to the subject. In embodiments, the compulsive disorder is obsessive compulsive disorder (OCD), gambling, aberrant sexual behavior, or combinations thereof.

In embodiments, the present disclosure provides methods for treating or preventing a personality disorder in a subject in need thereof, the methods comprising administering a therapeutically effective amount of a crystalline form of N,N-DMT disclosed herein (e.g., Form III and/or Form IV) or a pharmaceutical composition thereof to the subject. In embodiments, the personality disorder is conduct disorder, antisocial personality, aggressive behavior, or combinations thereof to the subject.

In embodiments, the present disclosure provides methods of treating or preventing PTSD, social anxiety disorder (e.g., social anxiety in autism spectrum disorder), autism spectrum disorder, binge eating disorder, alcohol use disorder, treatment resistant depression, major depressive disorder, generalized anxiety disorder, schizophrenia, borderline personality disorder, opioid use disorder, narcissistic personality disorder, avoidant personality disorder, tinnitus, anorexia nervosa, substance use disorder, chronic pain, tobacco addiction, bulimia nervosa, antisocial personality disorder, ADHD, a traumatic brain injury, body dysmorphia, hypoactive sexual desire disorder, migraines, agoraphobia, narcolepsy, obsessive compulsive disorder, and/or fibromyalgia in a subject in need thereof, the methods comprising administering a therapeutically effective amount of a crystalline form of N,N-DMT disclosed herein (e.g., Form III and/or Form IV) or a pharmaceutical composition thereof to the subject.

### EXAMPLES

The following example details the preparation and isolation of crystalline forms of N,N-DMT (e.g., Form III and Form IV). The study shows that Form III of N,N-DMT is metastable across all temperatures and to date, Form IV is the most thermodynamically stable polymorph of N,N-DMT having the highest known melting point.

I. Screening Experiments

Screening experiments were performed to identify stable and polymorphic forms of N,N-DMT. The experiments included an exploration of a variety of solvent systems, temperatures, crystallization conditions, and starting materials. Crystallization techniques employed included: slurrying, evaporation, pH swing, vapor diffusion, antisolvent precipitation, vapor stressing and heating. Each of these techniques are explained in greater detail below.

Slurrying: Suspensions were prepared by adding enough solids to a given solvent at the stated conditions so that undissolved solids were present. The mixture was then agitated (typically by stirring or oscillation) in a sealed vial at a given temperature for an extended period of time.

Evaporation: For fast evaporation, solutions were prepared in various solvents and, typically, filtered through a 0.2-μm nylon or PTFE filter. Each solution was allowed to evaporate from an open vial at ambient conditions, unless otherwise stated. For slow evaporation, solutions were prepared in various solvents and, typically, filtered through a 0.2-μm nylon or PTFE filter. Each solution was allowed to evaporate from a covered vial (such as loosely capped or covered with perforated aluminum foil) at ambient conditions.

pH swing experiments were conducted by combining N, N-DMT solids with water with stirring at ambient temperature, resulting in a solution with undissolved solids present. Aliquots of strong acid (e.g., 0.1 N HCIl) were added with stirring, resulting in a clear solution. Aliquots of strong base (e.g., 0.1 N NaOH) were added with stirring, resulting in white suspended solids. In some cases, needles, blades, and aggregates were observed. Crystals were culled and analyzed.

Vapor diffusion: Concentrated solutions were prepared in various solvents and, typically, filtered through a 0.2 μm nylon or PTFE filter. The filtered solution was dispensed into a small vial, which was then placed inside a larger vial containing antisolvent. The small vial was left uncapped, and the larger vial was capped to allow vapor diffusion to occur.

Antisolvent precipitation: Solutions were prepared in various solvents and aliquots of various antisolvents were dispensed with stirring until precipitation occurred. Mixtures were allowed to stir at specified conditions.

Vapor stressing: A small vial containing a given material was placed inside a larger vial containing solvent. The small vial was left uncapped, and the larger vial was capped to allow vapor stressing to occur at the stated temperature.

Heating: Conducted either by DSC (for examination of the thermograms and/or recovery of heated solids for XRPD) or heating on a hot plate/hot bench.

Three anhydrous/unsolvated polymorphs were observed: Forms I, III and IV (Form II reported in the literature was not observed in the screen). A comparison of the properties of the three polymorphic forms of N,N-DMT is provided in Table 1.

TABLE 1
Comparison of the Properties Form I, II, and IV of N,N-DMT

| Characteristics | Form I | Form III | Form IV |
|---|---|---|---|
| Physical appearance | White solids | White to off-white | White to off-white |
| | | solids | solids |
| Composition | Successfully indexed, | Pattern could not be | Successfully indexed, |
| [XRPD] | single crystalline | indexed as additional | consistent with |
| | phase, consistent with | minor peaks observed, | unsolvated N,N-DMT |
| | anhydrous/ | except a pattern was | |
| | unsolvated N,N-DMT | obtained from a multi- | |
| | Form I in literature | step heating | |
| | | experiment, which | |
| | | needed | |
| | | recrystallization of | |
| | | melted material | |
| | | at −20° C. | |
| Composition | Consistent with N,N- | Consistent w/N,N- | Consistent with N,N- |
| [¹H NMR] | DMT, no organic | DMT, negligible | DMT, no organic |
| | solvent | organic solvent | solvent |
| Melting point (° C.) | (a) DSC 10° C./min: | (a) DSC 10° C./min: | (a) DSC 10° C./min: |
| [DSC] | endo at 60° C. | endo at 44° C. | endo at 71° C. |
| | (onset 58° C.) | (onset 39° C.) | (onset 69° C.) |
| | (b) DSC 2° C./min: | (b) DSC 2° C./min: | (b) DSC 2° C./min: |
| | endo at 59° C. | sharp endo 68° C. | endo 70° C. |
| | (onset 58° C.) | (onset 67° C.) | (onset 69° C.) |
| Aqueous solubility | Low | Not determined | Low |
| (mg/mL)* | (<1) | | (<1) |
| Residual solvent/H₂O | 1.0% weight loss up | 1.4% weight loss | 1.1% weight loss |
| [TGA] | to 240° C. | 41° C.-201° C. | 50-223° C. |
| | Decomposition, | | |
| | evidenced by a steep | | |
| | drop in the TGA | | |
| | thermogram, begins | | |
| | above approx. 240° C. | | |
| Hygroscopicity | 0.04 wt % water at | Not determined | — |
| [DVS] | 60% RH and 0.18% | | |
| | water at 90% RH; no | | |
| | hysteresis observed | | |

Note:
*Solubilities are calculated based on the total solvent used to give a solution; actual solubilities may be greater because of the volume of the solvent portions used or a slow rate of dissolution. Values are rounded to the nearest whole number. If dissolution did not occur as determined by visual assessment, the value is reported as “<”.

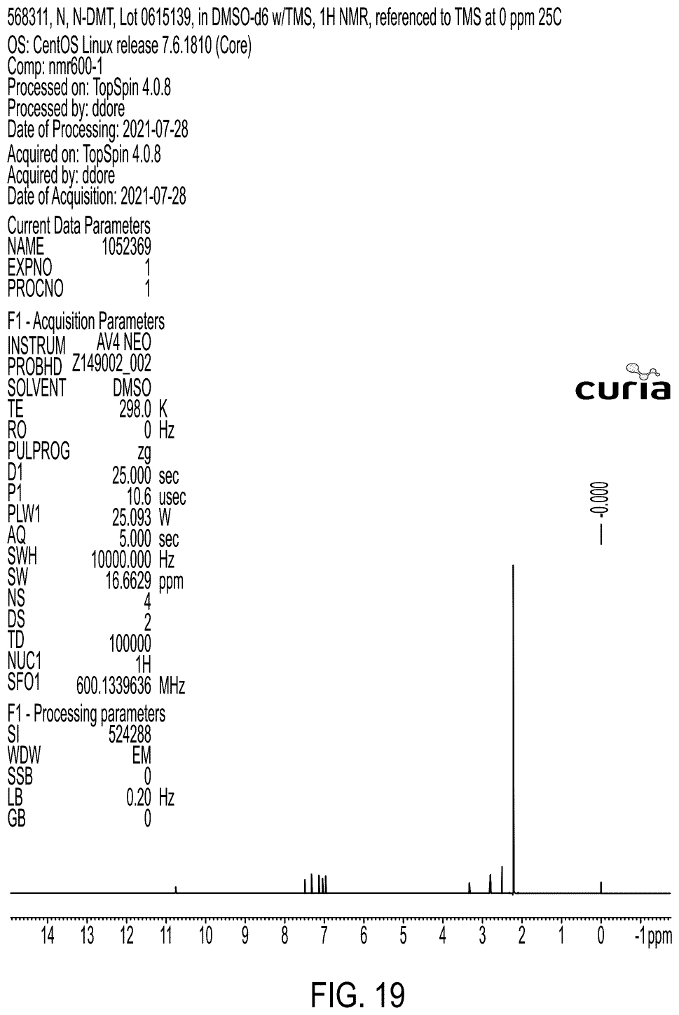

FIG. 19 shows a 1H NMR for N,N-DMT. The 1H NMR spectrum for each of the N,N-DMT samples disclosed herein was substantially the same as shown in FIG. 19.

II. X-ray Powder Diffraction (XRPD) Experiments

XRPD analysis was carried out in transmission geometry and reflection geometry.

XRPD in Transmission Geometry: XRPD patterns were collected with a PANalytical X′Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X′Celerator) located 240 mm from the specimen and Data Collector software v. 5.5. The data acquisition parameters for each pattern are displayed above the image in the Data section of this report. All images have the instrument labeled as X′Pert PRO MPD regardless of the instrument used.

XRPD in Reflection Geometry

XRPD patterns were collected with a PANalytical X′Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640f) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X′Celerator) located 240 mm from the sample and Data Collector software v. 5.5. The data acquisition parameters for each pattern are displayed above the image in the Data section of this report including the divergence slit (DS) and the incident-beam SS.

XRPD peak positions for Forms I, III & IV of N,N-DMT were determined and are enumerated below.

Form I

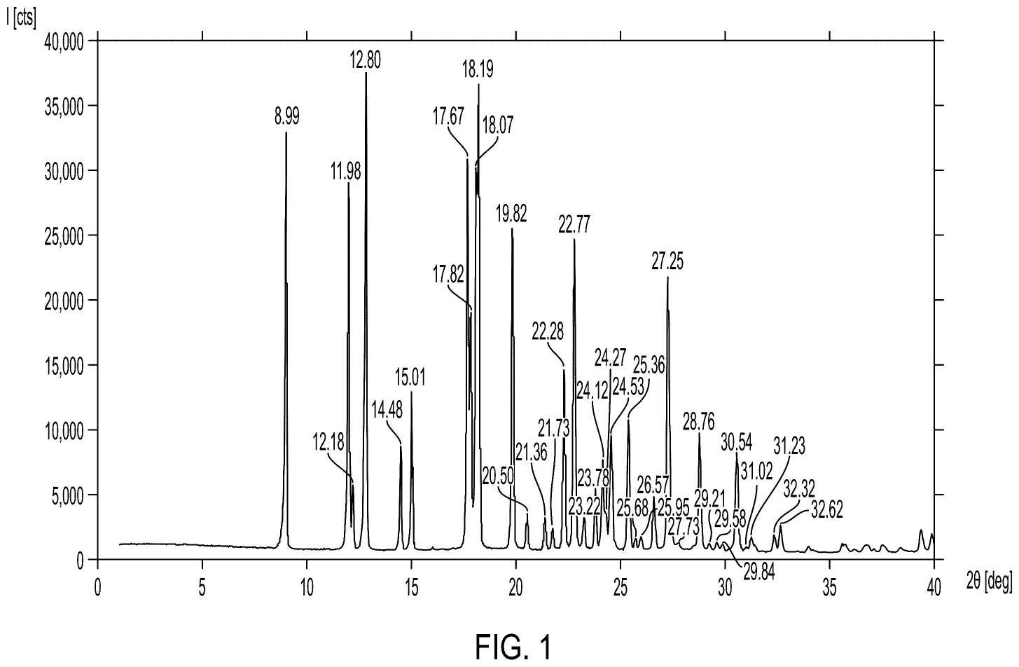

The XRPD pattern of Form I is shown in FIG. 1. Preferred orientation and particle statistic effects were assessed through comparison to the calculated XRPD pattern from the single crystal structure determination and determined to be negligible.

Observed diffraction peaks are shown in FIG. 1 and enumerated in Table 2, and representative peaks are listed in Table 3.

TABLE 2
Observed Diffraction Peaks for Form I

| °2θ | d space (Å) | Intensity (%) |
|---|---|---|
| 8.99 ± 0.2 | 9.829 ± 0.218 | 88 |
| 11.98 ± 0.2 | 7.382 ± 0.123 | 77 |
| 12.18 ± 0.2 | 7.261 ± 0.119 | 15 |
| 12.80 ± 0.2 | 6.910 ± 0.108 | 100 |
| 14.48 ± 0.2 | 6.112 ± 0.084 | 23 |
| 15.01 ± 0.2 | 5.898 ± 0.078 | 35 |
| 17.67 ± 0.2 | 5.015 ± 0.056 | 83 |
| 17.82 ± 0.2 | 4.973 ± 0.055 | 51 |
| 18.07 ± 0.2 | 4.905 ± 0.054 | 81 |
| 18.19 ± 0.2 | 4.873 ± 0.053 | 98 |
| 19.82 ± 0.2 | 4.476 ± 0.045 | 68 |
| 20.50 ± 0.2 | 4.329 ± 0.042 | 9 |
| 21.36 ± 0.2 | 4.156 ± 0.038 | 9 |
| 21.73 ± 0.2 | 4.087 ± 0.037 | 7 |
| 22.28 ± 0.2 | 3.987 ± 0.035 | 39 |
| 22.77 ± 0.2 | 3.902 ± 0.034 | 66 |
| 23.22 ± 0.2 | 3.828 ± 0.033 | 9 |
| 23.78 ± 0.2 | 3.739 ± 0.031 | 15 |
| 24.12 ± 0.2 | 3.687 ± 0.030 | 21 |
| 24.27 ± 0.2 | 3.664 ± 0.030 | 10 |
| 24.53 ± 0.2 | 3.626 ± 0.029 | 26 |
| 25.36 ± 0.2 | 3.509 ± 0.027 | 29 |
| 25.68 ± 0.2 | 3.466 ± 0.027 | 5 |
| 25.95 ± 0.2 | 3.431 ± 0.026 | 5 |
| 26.57 ± 0.2 | 3.352 ± 0.025 | 13 |
| 27.25 ± 0.2 | 3.270 ± 0.024 | 59 |
| 27.73 ± 0.2 | 3.215 ± 0.023 | 4 |
| 28.76 ± 0.2 | 3.102 ± 0.021 | 26 |
| 29.21 ± 0.2 | 3.055 ± 0.020 | 3 |
| 29.58 ± 0.2 | 3.017 ± 0.020 | 4 |
| 29.84 ± 0.2 | 2.992 ± 0.020 | 3 |
| 30.54 ± 0.2 | 2.925 ± 0.019 | 22 |
| 31.02 ± 0.2 | 2.881 ± 0.018 | 3 |
| 31.23 ± 0.2 | 2.862 ± 0.018 | 5 |
| 32.32 ± 0.2 | 2.768 ± 0.017 | 5 |
| 32.62 ± 0.2 | 2.743 ± 0.016 | 7 |

TABLE 3
Representative Diffraction Peaks for Form I

| °2θ | d space (Å) | Intensity (%) |
|---|---|---|
| 8.99 ± 0.2 | 9.829 ± 0.218 | 88 |
| 11.98 ± 0.2 | 7.382 ± 0.123 | 77 |
| 12.80 ± 0.2 | 6.910 ± 0.108 | 100 |
| 14.48 ± 0.2 | 6.112 ± 0.084 | 23 |
| 15.01 ± 0.2 | 5.898 ± 0.078 | 35 |
| 17.67 ± 0.2 | 5.015 ± 0.056 | 83 |
| 17.82 ± 0.2 | 4.973 ± 0.055 | 51 |
| 18.07 ± 0.2 | 4.905 ± 0.054 | 81 |
| 18.19 ± 0.2 | 4.873 ± 0.053 | 98 |
| 19.82 ± 0.2 | 4.476 ± 0.045 | 68 |
| 22.28 ± 0.2 | 3.987 ± 0.035 | 39 |
| 22.77 ± 0.2 | 3.902 ± 0.034 | 66 |
| 24.53 ± 0.2 | 3.626 ± 0.029 | 26 |
| 25.36 ± 0.2 | 3.509 ± 0.027 | 29 |
| 27.25 ± 0.2 | 3.270 ± 0.024 | 59 |
| 28.76 ± 0.2 | 3.102 ± 0.021 | 26 |
| 30.54 ± 0.2 | 2.925 ± 0.019 | 22 |

Form III

Preferred orientation and particle statistic effects were assessed through comparison to XRPD patterns obtained from diffractometers in different geometries and found to be significant. Observed diffraction peaks are shown in FIG. 2 and enumerated in Table 4, and prominent diffraction peaks are listed in Table 5. Note that none of the peaks are known to be representative or characteristic of this material since the state of preferred orientation in this sample is not known.

TABLE 4
Observed Diffraction Peaks for Form III

| °2θ | d space (Å) | Intensity (%) |
|---|---|---|
| 7.62 ± 0.2 | 11.592 ± 0.304 | 76 |
| 15.24 ± 0.2 | 5.809 ± 0.076 | 100 |
| 16.81 ± 0.2 | 5.270 ± 0.062 | 2 |
| 19.22 ± 0.2 | 4.614 ± 0.048 | 9 |
| 19.59 ± 0.2 | 4.528 ± 0.046 | 5 |
| 20.05 ± 0.2 | 4.425 ± 0.044 | 1 |
| 20.43 ± 0.2 | 4.344 ± 0.042 | 2 |
| 20.68 ± 0.2 | 4.292 ± 0.041 | 2 |
| 21.48 ± 0.2 | 4.134 ± 0.038 | 1 |
| 22.41 ± 0.2 | 3.964 ± 0.035 | 2 |
| 22.93 ± 0.2 | 3.875 ± 0.033 | 7 |
| 23.14 ± 0.2 | 3.841 ± 0.033 | 2 |
| 26.29 ± 0.2 | 3.387 ± 0.025 | 1 |
| 27.14 ± 0.2 | 3.283 ± 0.024 | 1 |
| 27.82 ± 0.2 | 3.204 ± 0.023 | 1 |
| 28.49 ± 0.2 | 3.130 ± 0.022 | 1 |
| 30.71 ± 0.2 | 2.909 ± 0.018 | 3 |
| 31.56 ± 0.2 | 2.833 ± 0.017 | 1 |

TABLE 5
Representative Diffraction Peaks for Form III

| °2θ | d space (Å) | Intensity (%) |
|---|---|---|
| 7.62 ± 0.2 | 11.592 ± 0.304 | 76 |
| 15.24 ± 0.2 | 5.809 ± 0.076 | 100 |
| 19.22 ± 0.2 | 4.614 ± 0.048 | 9 |
| 19.59 ± 0.2 | 4.528 ± 0.046 | 5 |
| 22.93 ± 0.2 | 3.875 ± 0.033 | 7 |

Form IV

Preferred orientation and particle statistic effects were assessed through comparison to the calculated XRPD pattern from the single crystal structure determination and determined to be negligible. Observed peaks are shown in FIG. 3 and enumerated in Table 6, and representative peaks are listed in Table 7.

TABLE 6
Observed Diffraction Peaks for Form IV

| °2θ | d space (Å) | Intensity (%) |
|---|---|---|
| 7.68 ± 0.2 | 11.502 ± 0.299 | 5 |
| 10.35 ± 0.2 | 8.540 ± 0.165 | 2 |
| 11.92 ± 0.2 | 7.419 ± 0.124 | 5 |
| 13.05 ± 0.2 | 6.779 ± 0.103 | 5 |
| 13.61 ± 0.2 | 6.501 ± 0.095 | 100 |
| 13.89 ± 0.2 | 6.370 ± 0.091 | 29 |
| 13.99 ± 0.2 | 6.325 ± 0.090 | 25 |
| 15.16 ± 0.2 | 5.840 ± 0.077 | 5 |
| 15.39 ± 0.2 | 5.753 ± 0.074 | 22 |
| 16.16 ± 0.2 | 5.480 ± 0.067 | 11 |
| 16.90 ± 0.2 | 5.242 ± 0.062 | 10 |
| 17.45 ± 0.2 | 5.078 ± 0.058 | 9 |
| 17.76 ± 0.2 | 4.990 ± 0.056 | 57 |
| 18.05 ± 0.2 | 4.911 ± 0.054 | 17 |
| 18.64 ± 0.2 | 4.756 ± 0.051 | 42 |
| 19.25 ± 0.2 | 4.607 ± 0.047 | 6 |
| 19.81 ± 0.2 | 4.478 ± 0.045 | 4 |
| 20.79 ± 0.2 | 4.269 ± 0.041 | 100 |
| 21.11 ± 0.2 | 4.205 ± 0.039 | 11 |
| 21.63 ± 0.2 | 4.105 ± 0.038 | 17 |
| 22.39 ± 0.2 | 3.968 ± 0.035 | 15 |
| 22.69 ± 0.2 | 3.916 ± 0.034 | 12 |
| 23.59 ± 0.2 | 3.768 ± 0.031 | 28 |
| 23.81 ± 0.2 | 3.734 ± 0.031 | 4 |
| 24.47 ± 0.2 | 3.635 ± 0.029 | 21 |
| 24.77 ± 0.2 | 3.591 ± 0.029 | 10 |
| 25.22 ± 0.2 | 3.528 ± 0.028 | 5 |
| 26.06 ± 0.2 | 3.417 ± 0.026 | 23 |
| 26.66 ± 0.2 | 3.341 ± 0.025 | 24 |
| 26.90 ± 0.2 | 3.312 ± 0.024 | 3 |
| 27.53 ± 0.2 | 3.237 ± 0.023 | 2 |
| 27.99 ± 0.2 | 3.185 ± 0.022 | 5 |
| 28.23 ± 0.2 | 3.159 ± 0.022 | 4 |
| 28.61 ± 0.2 | 3.118 ± 0.021 | 3 |
| 28.75 ± 0.2 | 3.103 ± 0.021 | 3 |
| 29.28 ± 0.2 | 3.048 ± 0.020 | 3 |
| 29.46 ± 0.2 | 3.030 ± 0.020 | 2 |
| 29.63 ± 0.2 | 3.013 ± 0.020 | 2 |
| 30.17 ± 0.2 | 2.960 ± 0.019 | 2 |
| 30.36 ± 0.2 | 2.942 ± 0.019 | 3 |
| 30.59 ± 0.2 | 2.920 ± 0.019 | 3 |
| 30.91 ± 0.2 | 2.891 ± 0.018 | 4 |
| 31.09 ± 0.2 | 2.874 ± 0.018 | 8 |
| 31.63 ± 0.2 | 2.826 ± 0.017 | 3 |
| 31.88 ± 0.2 | 2.805 ± 0.017 | 4 |
| 32.83 ± 0.2 | 2.726 ± 0.016 | 2 |
| 33.21 ± 0.2 | 2.695 ± 0.016 | 2 |
| 33.74 ± 0.2 | 2.654 ± 0.015 | 2 |
| 34.45 ± 0.2 | 2.601 ± 0.015 | 2 |
| 35.31 ± 0.2 | 2.540 ± 0.014 | 2 |

TABLE 7
Representative Diffraction Peaks for Form IV

| °2θ | d space (Å) | Intensity (%) |
|---|---|---|
| 13.61 ± 0.2 | 6.501 ± 0.095 | 100 |
| 13.89 ± 0.2 | 6.370 ± 0.091 | 29 |
| 13.99 ± 0.2 | 6.325 ± 0.090 | 25 |
| 15.39 ± 0.2 | 5.753 ± 0.074 | 22 |
| 17.76 ± 0.2 | 4.990 ± 0.056 | 57 |
| 18.64 ± 0.2 | 4.756 ± 0.051 | 42 |
| 20.79 ± 0.2 | 4.269 ± 0.041 | 100 |
| 23.59 ± 0.2 | 3.768 ± 0.031 | 28 |
| 24.47 ± 0.2 | 3.635 ± 0.029 | 21 |
| 26.06 ± 0.2 | 3.417 ± 0.026 | 23 |
| 26.66 ± 0.2 | 3.341 ± 0.025 | 24 |

Single Crystal XRD Experiments

Single crystal structures were afforded for Forms I and IV as described below.

Preparation of Samples for Single Crystal XRD:

Form I: N,N-DMT solids (83.7 mg) were combined with water (2 mL) with stirring at ambient temperature, resulting in a solution with undissolved solids present. Aliquots of 0.1 N HCI, totaling 10.5 mL, were added with stirring, resulting in a clear solution. Aliquots of 0.1 N NaOH, totaling 11.5 mL, were added with stirring, resulting in white suspended solids. Needles, blades, and aggregates were observed. A crystal was culled and analyzed.

Form IV: N,N-DMT solids (102.01 mg) were combined with heptane (1 mL) with stirring at ˜40° C., resulting in a solution with undissolved solids present. The slurry was left to stir at ˜40° C. for 12 days, after which thick birefringent needles were observed. A crystal was culled and analyzed.

Single Crystal XRD Data Collection:

Form I: A colorless block having approximate dimensions of 0.18×0.11×0.06 mm³, was mounted on a polymer loop in random orientation. Preliminary examination and data collection were performed on a Rigaku SuperNova diffractometer, equipped with a copper anode microfocus sealed X-ray tube (Cu Kα λ=1.54184 Å) and a Dectris Pilatus3 R 200K hybrid pixel array detector.

Cell constants and an orientation matrix for data collection were obtained from least-squares refinement using the setting angles of 2590 reflections in the range 4.4940°θ<0 <75.3650°. The

space group was determined by the program CRYSALISPRO to be P21/c (international tables no. 14). The data were collected to a maximum diffraction angle (2θ) of 151.432° at room temperature.

Form IV: A colorless block having approximate dimensions of 0.17×0.15×0.06 mm³, was mounted on a polymer loop in random orientation. Preliminary examination and data collection were performed on a Rigaku SuperNova diffractometer, equipped with a copper anode microfocus sealed X-ray tube (Cu Kα λ=1.54184 Å) and a Dectris Pilatus3 R 200K hybrid pixel array detector.

Cell constants and an orientation matrix for data collection were obtained from least-squares refinement using the setting angles of 5465 reflections in the range 3.8290°θ<0 <74.9960° . The

space group was determined by the program CRYSALISPRO to be P21/n (international tables no. 14). The data were collected to a maximum diffraction angle (2θ) of 151.584° at room temperature.

The single crystal data obtained from Forms I and IV are enumerated below in Table 8.

TABLE 8
Single Crystal Data of Polymorphic Forms I & IV of N,N-DMT

| Crystal data | Form I | Form IV |
|---|---|---|
| Crystal system | monoclinic | monoclinic |
| Space group | P2₁/c | P2₁/n |
| Cell parameters | a = 7.4379(2) Å | a = 7.8301(2) Å |
| | b = 19.6312(4) Å | b = 22.9828(4) Å |
| | c = 7.8759(2) Å | c = 12.7380(2) Å |
| | α = 90° | α = 90° |
| | β = 97.857(3)° | β = 91.980(2)° |
| | γ = 90° | γ = 90° |
| Unit cell volume | 1139.20(5) | 2290.93(8) |
| (Å3) | | |

Note:
Standard uncertainty is written in crystallographic parenthesis notation, e.g. 0.123(4) is equivalent to 0.123 ± 0.004.

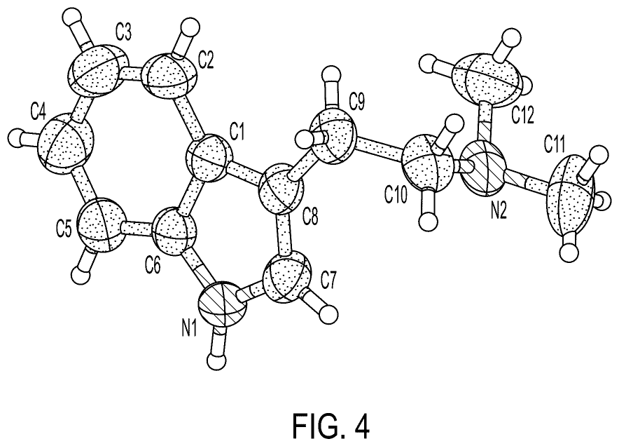

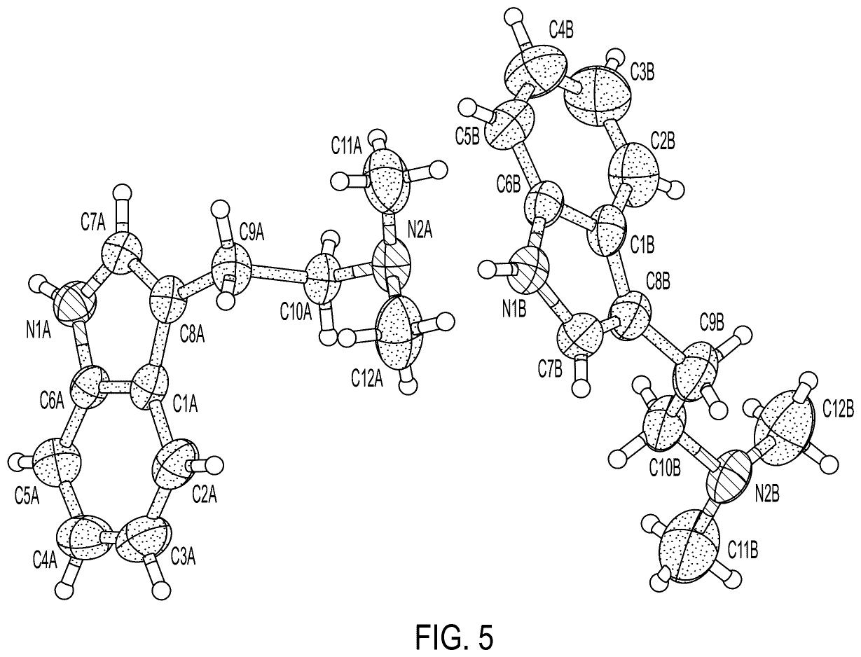

Atomic displacement ellipsoid diagrams of N,N-DMT for Form I and Form IV are shown in FIG. 4 and FIG. 5, respectively.

IV. Thermal Behavior Experiments

Differential Scanning Calorimetry (DSC):

DSC was performed using a Mettler-Toledo DSC3+ differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, the weight was accurately recorded, and the sample was inserted into the DSC cell. A weighed aluminum pan configured as the sample pan was placed on the reference side of the cell. The pan lid was pierced prior to sample analysis. The samples were analyzed from −25° C. to 250° C. at 10° C./min unless otherwise stated.

Thermogravimetric and Differential Scanning Calorimetry (TGA or TGA/DSC)

TGA or TGA/DSC analyses were performed using a Mettler-Toledo TGA/DSC3+ analyzer. Temperature and enthalpy adjustments were performed using indium, tin, zinc, and phenyl salicylate, and then verified with indium. The balance was verified with calcium oxalate. The sample was placed in an aluminum pan. The pan was hermetically sealed, the lid pierced, and the pan was then inserted into the TG furnace. A weighed aluminum pan configured as the sample pan was placed on the reference platform. The furnace was heated under nitrogen. Samples were analyzed from 25° C. to 350° C. at 10° C./min. Thermogravimetric analyses typically experience a period of equilibration at the start of each analysis, indicated by parentheses on the thermograms in the Figures section (V). The starting temperature for relevant weight loss calculations is selected at a point beyond this region (typically above 35° C.) for accuracy.

Form I:

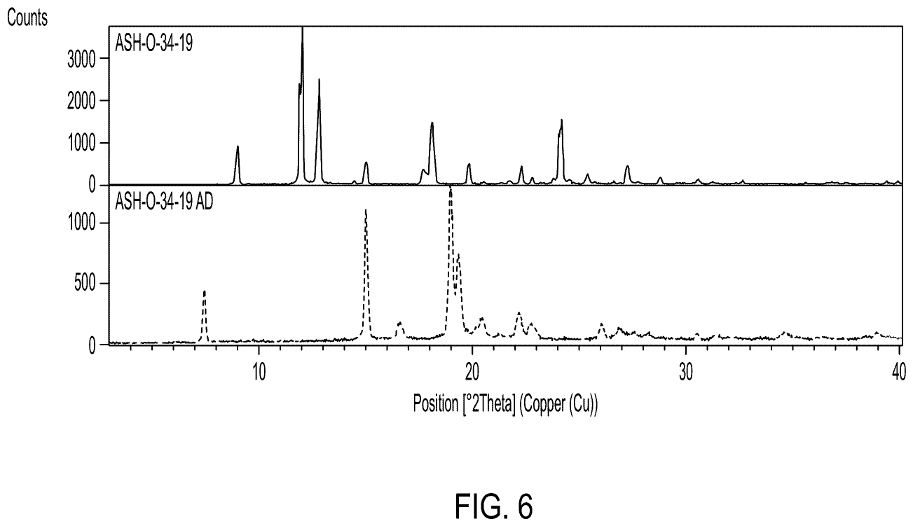

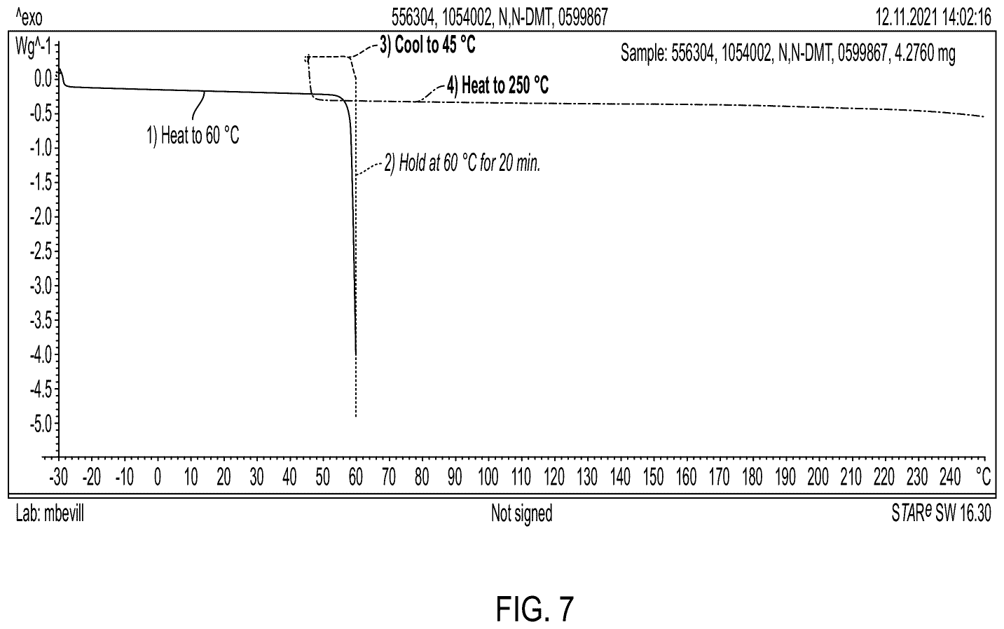

A DVS cycle study on N,N-DMT Form I had exhibited a form change when dried at 60° C. (as a final step of drying the API during manufacture) (FIG. 6). To study this behavior further, solid state cycling DSC thermograms for Form I were collected (FIG. 7). FIG. 7 is a thermogram for a sample that was initially heated to 60° C., causing the solids to melt. The melted material was held at 60° C.for 20 minutes, then cooled to 45° C. and reheated to 250° C. No significant events, such as recrystallization or melting of a higher-melting form, were observed.

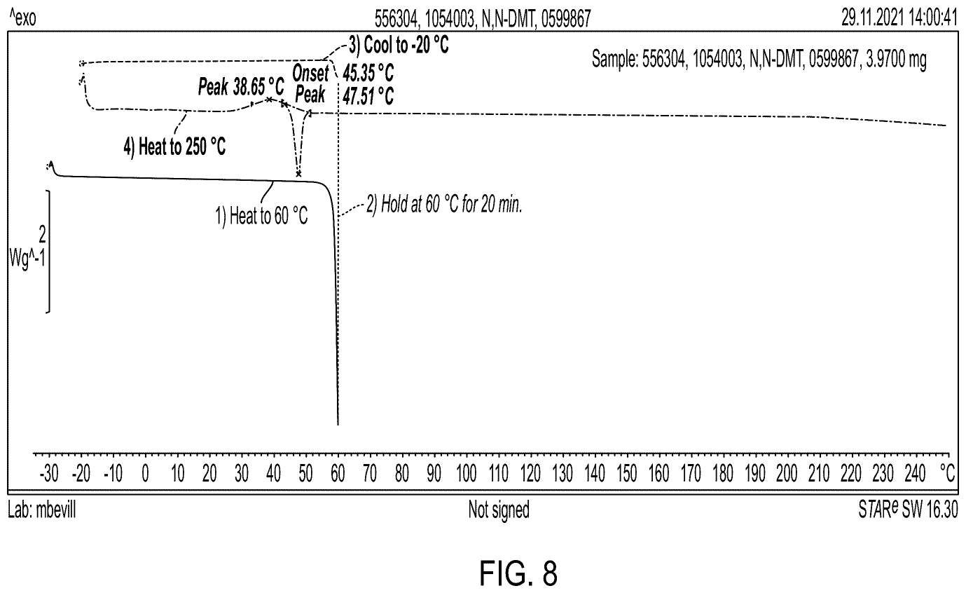

A similar experiment was conducted, but the melted material was cooled down to −20° C. before being reheated to 250° C.(FIG. 8). In this experiment, an exotherm is observed around 39° C. in the final heating step, consistent with crystallization, and is followed immediately by an endotherm at approximately 45° C. (onset), indicative of melting. This melting point is similar to that observed for Form III. The DSC experiments indicate that drying of the post-DVS solids at 60° C. melted the material, and then it recrystallized upon cooling to ambient temperature to lower-melting Form III.

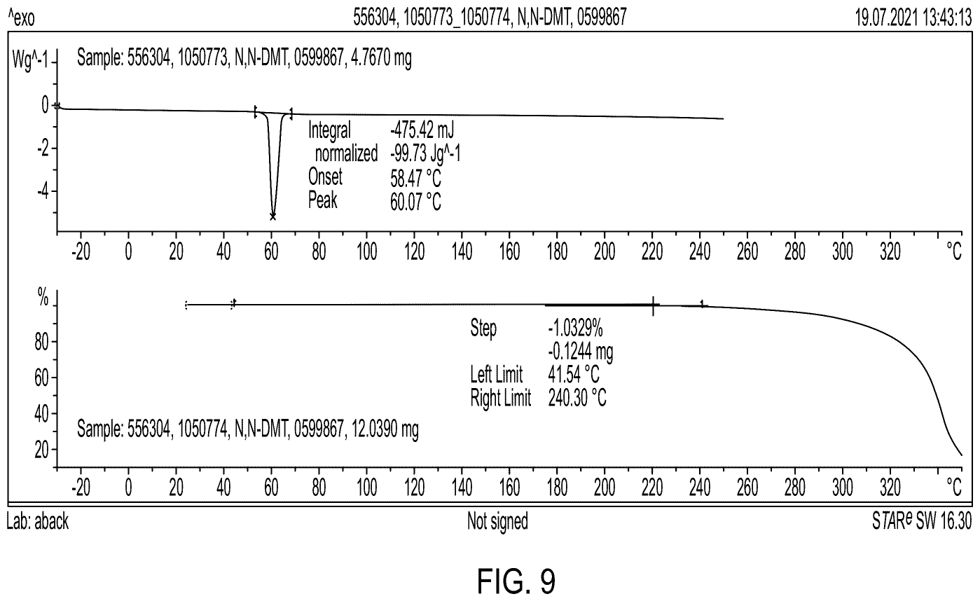

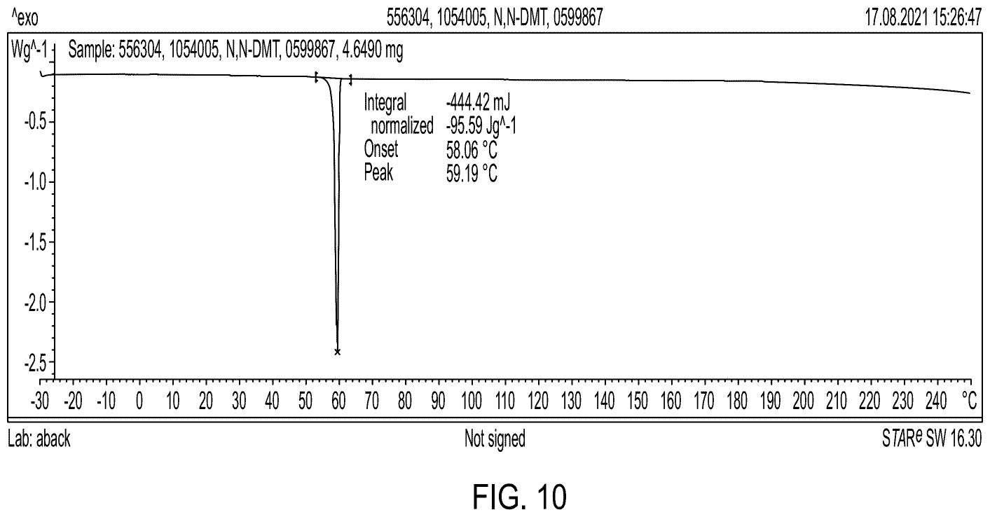

DSC and TGA thermograms for Form I were also collected at the standard 10° C./minute heating rate (FIG. 9). An additional DSC thermogram was obtained at a slower heating rate (2° C./minute) to obtain more sensitive data (FIG. 10).

A melting endotherm is observed in both thermograms at 58° C.(onset), consistent with the melting point reported for Form I in literature. The TGA thermogram exhibits only 1.0% weight loss up to 240° C., consistent with an anhydrous/unsolvated material. Decomposition, evidenced by a steep drop in the TGA thermogram, begins above approximately 240° C.

Form III:

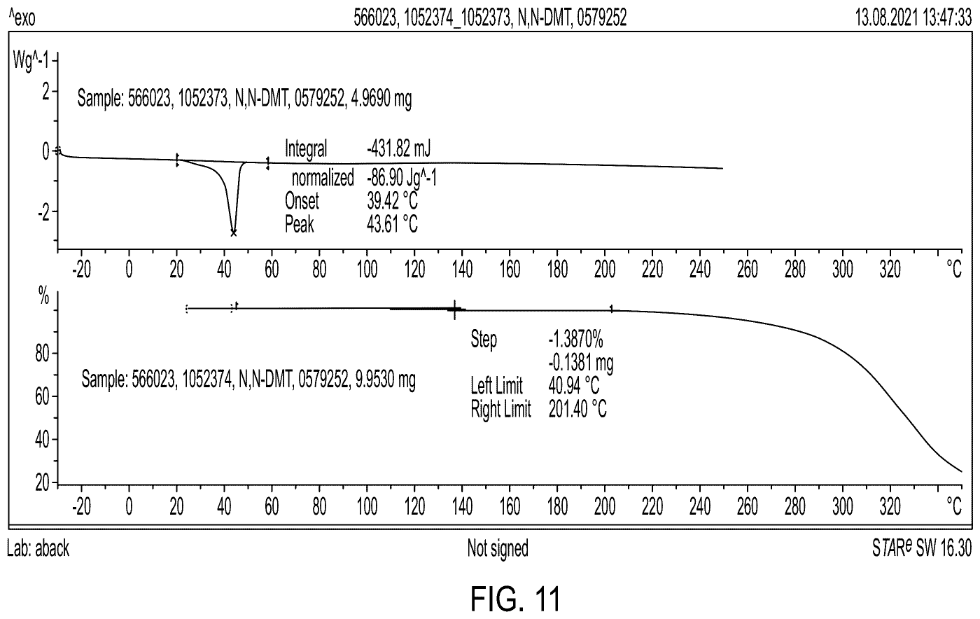

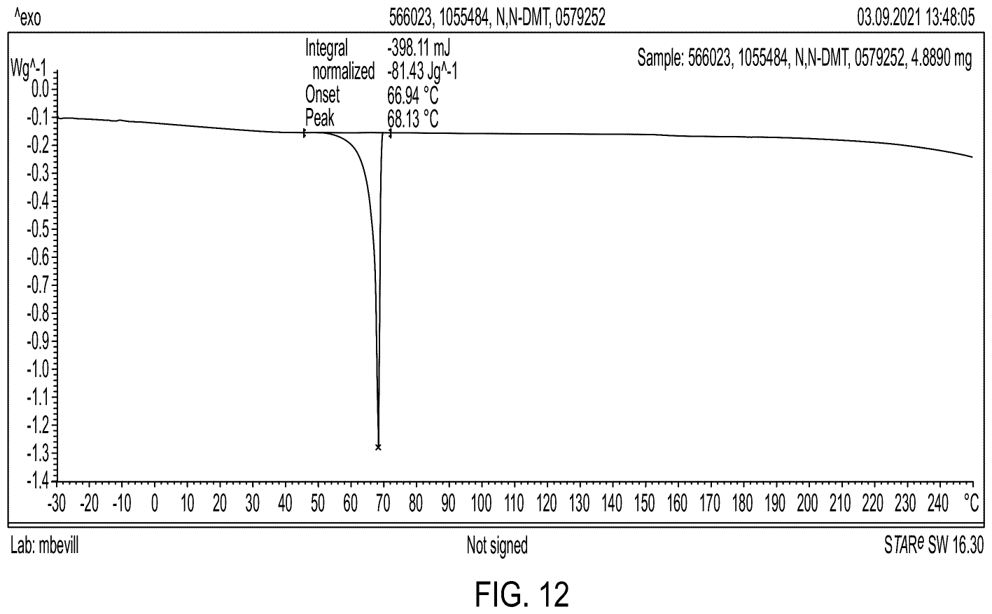

DSC and TGA thermograms, collected at a heating rate of 10° C. per minute (FIG. 11). A melting endotherm is observed by DSC at 39° C. (onset). Negligible weight loss by TGA up to 201° C. is consistent with an anhydrous/unsolvated material. Decomposition is observed above approximately 240 ° ° C. To obtain more sensitive data, DSC thermograms were collected at a heating rate of 2° C. per minute (FIG. 12). In FIG. 12, when the ramping rate was brought down to 2C/ min, the crystallization (into another form) and melt of form III are simultaneous due to the low temp ramping rate. Based on the endotherm with a melting onset at 66.94° C., we can conclude that form III had time to melt then recrystallize into form IV which melted around 67° C.

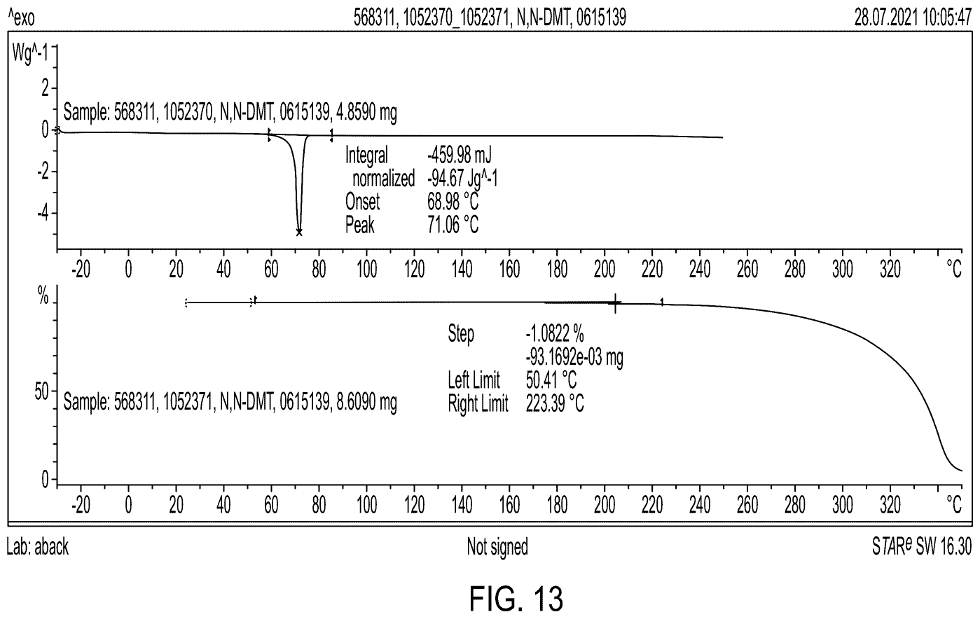

Solid state heating experiments were also conducted starting with Form III (+ a minor unknown component) (FIG. 13). These experiments were designed to observe melting and recrystallization behavior at a variety of temperatures. Approximate melt onset temperature for Form III (39° C.) from DSC characterization was used as a basis for determining form conversions.

Form III was observed to concurrently melt and recrystallize to Form IV when held between 35° C. and 40° C. However, when melted material was cooled to RT or −20° C., it recrystallized to Form III in some cases. In one experiment, melted material did not exhibit signs of crystallization upon standing in the freezer for 1 day, so heptane was added, and the sample was left to stir in the freezer for 12 days, resulting in crystallization to Form IV.

Form IV:

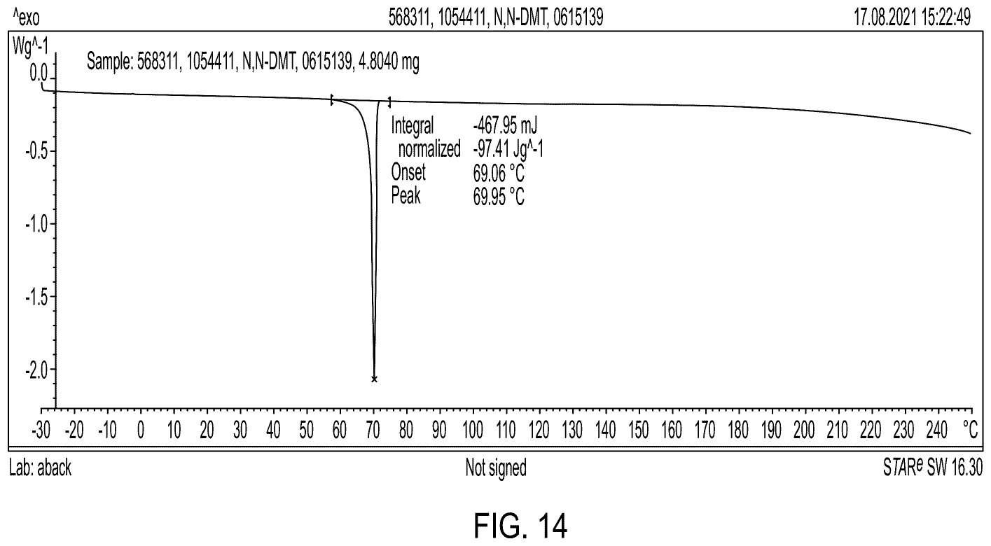

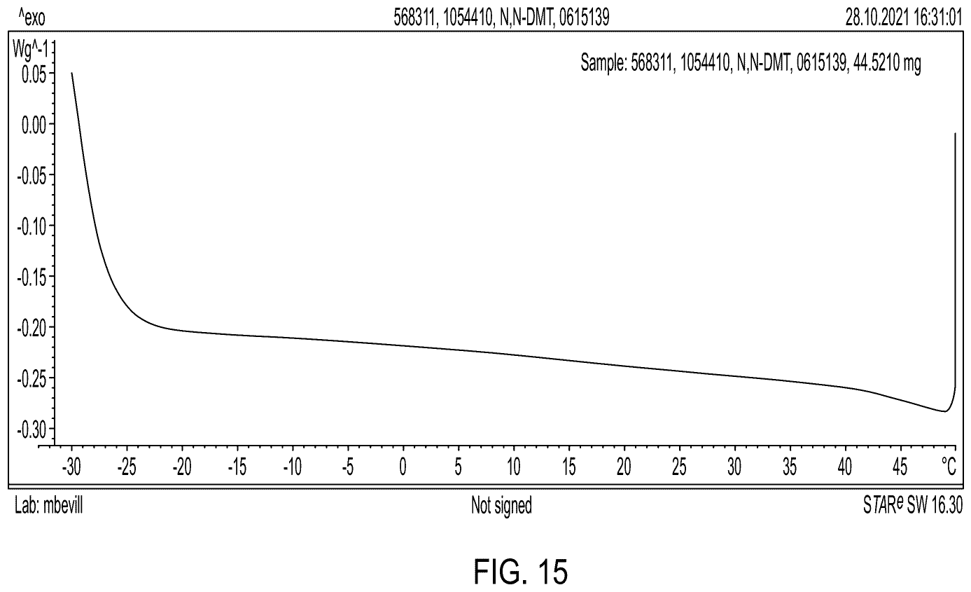

DSC and TGA thermograms were collected at a heating rate of 10° C./min, and an additional DSC thermogram was obtained at 2° C./min (FIG. 14 and FIG. 15, respectively). Both DSC thermograms exhibit a sharp melting endotherm at an onset of 69° C. Negligible weight loss up to 223° ° C. by TGA is consistent with an anhydrous/unsolvated material. Decomposition is observed above approximately 240° C.

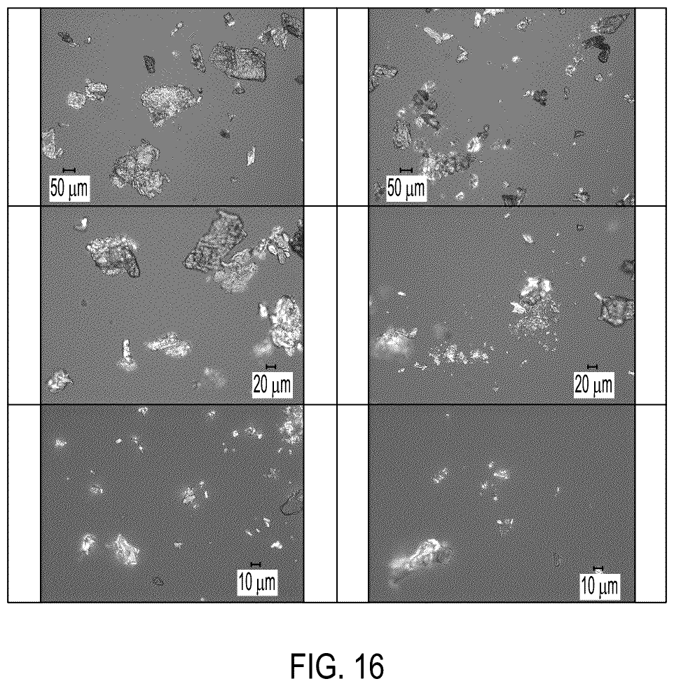

Solid state heating experiments were conducted starting with Form IV (FIG. 16). As for Form III, these experiments were designed to observe melting and recrystallization behavior at a variety of temperatures and, the approximate melt onset temperature for Form IV (69° C.) from DSC characterization was used as a basis for determining form conversions. Form IV exhibited no changes upon heating at 50° C.for 10 minutes.

V. Stability Experiments to Determine Relative Stability of Forms I, III, and IV

Slurry experiments were conducted to determine the relative stability of Forms I, III, and IV. The screening conditions are tabulated at Tables 9-11.

TABLE 9
Stable Form Screen Slurries of N,N-DMT of Form I

| | | | XRPD¹ |
|---|---|---|---|
| Solvent | Conditions | Observations | Result |
| acetone/water | 5° C., 13 d | white solids, fines | Form IV |
| (10:90) | | and aggregates, B/E | |
| | attempted slurry, | clear soln. w/brown | Form I + |
| | RT, 13 d | clear liquid | minor Form IV |
| cyclohexane | RT, 13 d | white solids, | Form I |
| | | needles/aggregates, | |
| | | B/E | |
| EtOAc/ | attempted slurry, | clear soln. w/brown | Form I |
| cyclohexane | RT, 13 d | clear liquid | |
| (5:95) | | | |
| heptane | RT, 13 d | white solids, fines, | Form I |
| | | B/E | |
| | 40° C., 13 d | white solids, thick | Form IV + |
| | | needles, B/E | minor Form I |
| | −20° C., 17 d | white solids, fines, | Form I |
| | | B/E | |
| IPA/water | attempted slurry, | clear yellow/orange | — |
| (5:95) | RT, 13 d | soln. | |
| IPA/water | 5° C., 13 d | white solids, needles | Form I |
| (10:90) | | and aggregates, B/E | |
| | attempted slurry, | clear soln. w/brown | Form IV + |
| | 40° C., 13 d | clear liquid | Form III |
| MeOH/water | attempted slurry, | clear soln. w/brown | Form IV + |
| (20:80) | RT, 13 d | clear liquid | minor Form I |
| THF/water | attempted slurry, | clear soln. w/brown | Form IV |
| (5:95) | RT, 13 d | clear liquid | |

¹Solids from RT slurries were analyzed while damp.

TABLE 10
Stable Form Screen Slurries of N,N-DMT of Form III

| | | | XRPD¹ |
|---|---|---|---|
| Solvent | Conditions | Observations | Result |
| acetone/water | attempted slurry, | yellow oil in soln. | — |
| (10:90) | RT, 15 d | | |
| EtOAc/heptane | −20° C., | white fines, B/E | Form I |
| (5:95) | 15 d | | |
| heptane | RT, 15 d | white solids, fines, | Form I |
| | | B/E | |
| IPAc/heptane | RT, 13 d | white solids, fines, | Form IV |
| (3:97) | | B/E | |
| MeOH/water | 5° C., 15 d | white fines, B/E² | amorphous |
| (20:80) | | | |
| — | sample stored at | off-white aggregated | Form I + |
| | ambient conditions | needles, some B/E | minor |
| | ~2 wks, then | | Form III |
| | observed and | | |
| | reanalyzed | | |

¹Solids from RT slurries were analyzed while damp.
²XRPD analyst noted that sample was oily before analysis.

TABLE 11
Stable Form Screen Slurries of N,N-DMT of Form IV

| | | | XRPD |
|---|---|---|---|
| Solvent | Conditions | Observations | Result |
| acetone/water | RT, 13 d | off-white solids, | Form IV |
| (10:90) | | fines, B/E | |
| EtOAc/heptane | −20° C., | off-white solids, | Form IV |
| (5:95) | 13 d | fines, B/E | |
| heptane | RT, 13 d | off-white solids, | Form IV |
| | | fines, B/E | |
| MeOH/water | 5° C., | off-white solids, | Form IV |
| (20:80) | 13 d | fines, B/E | |

In these experiments, saturated solutions containing excess undissolved solids were stirred for extended durations. At these conditions, a metastable form would dissolve at a concentration that is supersaturated with respect to the stable form, causing crystallization of the more stable form over time. Slurries were conducted RT, 40° C., 5° C., and −20° C.for approximately 2 weeks. Solvent systems for which the compound was expected to exhibit limited solubility were employed in an effort to provide suitable conditions for conversion to a more stable form. It should be noted that even with these efforts, a lack of form change does not conclusively determine the stable form and could be attributed to insufficient solubility or time for conversion to a more stable form.

The observed form conversions suggest that Form IV is the most thermodynamically stable polymorph. Form IV also remained unchanged upon slurrying in various solvent systems. All slurry experiments starting with Form III converted to other forms, indicating it is metastable at all conditions tested. Additionally, conversion of Form III to Form I at −20° C.and RT suggests that Form I is likely more stable than Form III within that temperature range. Additionally, Form III exhibited a tendency to crystallize in mixtures with a minor unknown component or other known forms throughout the screen, consistent with its metastable nature.

Since Forms I and IV were identified as two stable forms, further interconversion slurries relative to each other were studied.

VI. Stability Experiments to Determine Relative Stability of Forms III and IV

Interconversion slurries were conducted in heptane at RT, −20° C., and 40° C. as in Table 12. In these slurries, the heptane was pre-saturated with N,N-DMT using Form IV solids at the stated temperature, and a portion of the liquid phase was filtered into a mixture of Form I and Form IV solids. A pre-saturated liquid phase is utilized to minimize any kinetic dissolution effects, allowing the less stable (and more soluble) form to dissolve and the most stable (and least soluble) form to precipitate.

TABLE 12
Slurry Competition Studies Between Forms I and IV

| | Starting Materials | | | |
|---|---|---|---|---|
| | | | | XRPD |
| Forms | Solvent | Conditions | Observations | Result |
| Form I | heptane | RT, 7 d | off-white solids, fines, | Form IV |
| and | | | B/E | |
| Form IV | heptane | −20° C., 7 d | off-white solids, fines, | Form IV |
| | | | B/E | |
| | heptane | 40° C., 7 d | off-white solids, fines, | Form IV |
| | | | B/E | |

Full conversion to Form IV was observed for all three slurries, indicating it is more stable at those conditions.

VI. Polarized light photomicrographs for Forms I and IV

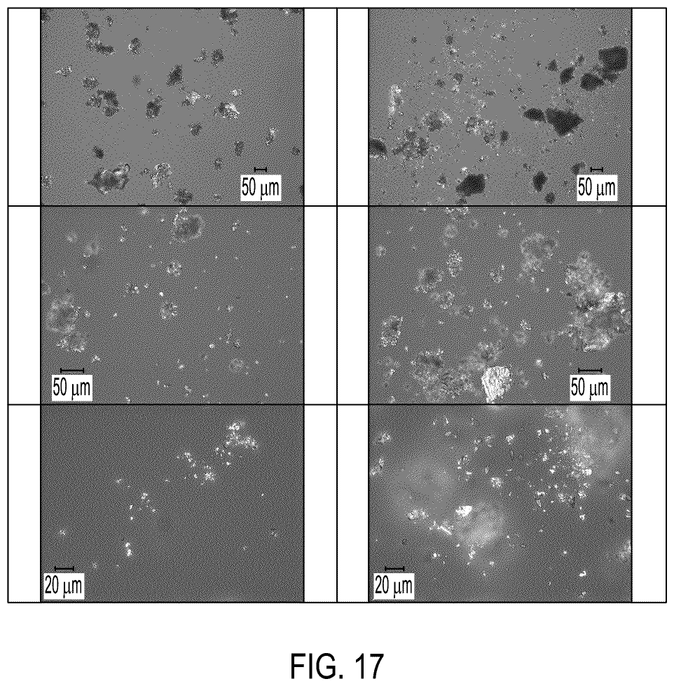

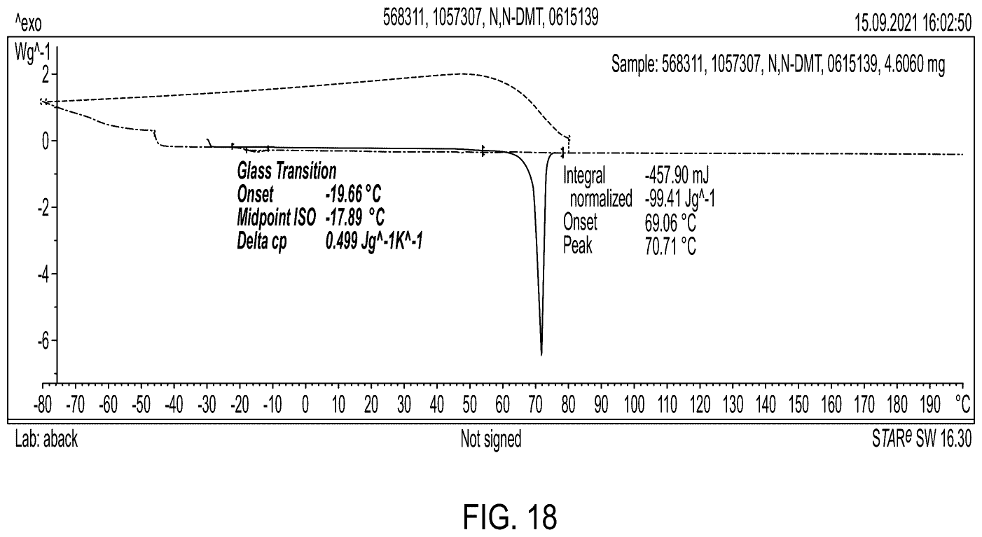

Polarized light microscopy for select forms (Forms I & IV) was performed using a Leica DM LP microscope equipped with a SPOT Insight™ color digital camera. Each sample was placed on a glass slide, a cover glass was placed over the sample, and a drop of mineral oil was added to cover the sample by capillarity. Each sample was observed with crossed polarizers and a first order red compensator. Images were captured using SPOT software (v. 4.5.9). A micron bar was inserted onto each image as a reference for particle size. The photomicrographs are shown in FIG. 17 (Form I) and (Form IV) FIG. 18 displaying agglomerates and individual particles consisting of blades, flakes, and anhedral and drusy particles.

VIII. Amorphous N,N-DMT

Attempts to generate amorphous material did not yield free-flowing amorphous solids, heating/melting experiments were conducted to determine the Tg. Observations from a heating/melting experiments indicated that amorphous solids readily crystallize at temperatures spanning −20° C. to 40° C. A cycling DSC experiment was performed to investigate a potential glass transition temperature by preparing amorphous solids in situ, starting with Form IV.

The thermogram is presented in FIG. 19. At the start of the experiment, Form IV solids were heated past the melt (to 80° C.) and held for 10 minutes. The melted material was quickly cooled to −80° C., then reheated to 200° C. A glass transition (T_{g}) is observed as a step in the green curve at −18 ° ° C. Amorphous materials tend to spontaneously crystallize at temperatures above the Tg. Such a low Tg for N,N-DMT is indicative of poor physical stability of amorphous material, particularly at temperatures above −18° C.

IX. Conclusion

The polymorph form, Form IV, identified in this study is found to be the most thermodynamically stable form, and resultantly exhibits a higher melting point than the other known polymorphic forms. On the contrary, the known polymorph, Form I, has been found to be less stable under various conditions (converts to Forms III & Form IV), making it less suitable as an API.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

1. Form IV of N,N-dimethyltryptamine.

2. Form IV of N,N-dimethyltryptamine of claim 1, characterized by peaks in an X-ray powder diffraction (XRPD) pattern at 13.6±0.2, 20.8±0.2, and 17.80±0.2 °2θ.

3. The Form IV of N,N-dimethyltryptamine of claims 2, further characterized by at least one XRPD peak selected from 13.9±0.2, 14.0±0.2, 15.4±0.2, 18.6±0.2, 23.6±0.2, 24.5±0.2, 26.0±0.2, or 26.7±0.2 °2θ.

4. canceled

5. canceled

6. canceled

7. canceled

8. canceled

9. canceled

10. canceled

11. canceled

12. canceled

13. canceled

14. The Form IV of N,N-dimethyltryptamine of claim 1, characterized by peaks in an XRPD pattern at 7.7±0.2, 10.4±0.2, 11.9±0.2, 13.0±0.2, 13.6±0.2, 13.9±0.2, 14.0±0.2, 15.2±0.2, 15.4±0.2, 16.2±0.2, 16.9±0.2, 17.5±0.2, 17.8±0.2, 18.1±0.2, 18.6±0.2, 19.3±0.2, 19.8±0.2, 20.8±0.2, 21.1±0.2, 21.6±0.2, 22.4±0.2, 22.7±0.2, 23.6±0.2, 23.8±0.2, 24.5±0.2, 24.8±0.2, 25.2±0.2, 26.1±0.2, 26.7±0.2, 26.9±0.2, 27.5±0.2, 28.0±0.2, 28.2±0.2, 28.6±0.2, 28.8±0.2, 29.3±0.2, 29.5±0.2, 29.6±0.2, 30.2±0.2, 30.4±0.2, 30.6±0.2, 30.9±0.2, 31.1±0.2, 31.6±0.2, 31.9±0.2, 32.8±0.2, 33.2±0.2, 33.7±0.2, 34.5±0.2, and 35.3±0.2 ° 20.

15. The Form IV of N,N-dimethyltryptamine of claim 1, characterized by an XRPD pattern substantially similar to that shown in FIG. 3.

15. canceled

17. The Form IV of N,N-dimethyltryptamine of any of claims 1-16, which exhibits a Differential Scanning Calorimetry (DSC) thermogram comprising an endothermic peak at 7015° C.

18. The Form IV of N,N-dimethyltryptamine of claim 1, which exhibits substantially no weight loss at a temperature under 225±5° C. as measured by thermogravimetric (TGA) analysis.

19. The Form IV of N,N-dimethyltryptamine of claim 1, having a melting point of 69±5° C.

20. Form III of N,N-dimethyltryptamine.

21. The Form III of N,N-dimethyltryptamine of claim 20, characterized by peaks in an X-ray powder diffraction (XRPD) pattern at 7.6±0.2 and 15.2±0.2.

22. The Form III of N,N-dimethyltryptamine of claims 21, further characterized by at least one XRPD peak selected from 19.2±0.2 °74, 19.6±0.2, or 23.0±0.2 °2θ.

23. canceled

24. canceled

25. canceled

26. canceled

27. The Form III of N,N-dimethyltryptamine of claim 20, characterized by peaks in an XRPD pattern at 7.6±0.2, 15.2±0.2, 16.8±0.2, 19.2±0.2, 19.6±0.2, 20.0±0.2, 20.4±0.2, 20.7±0.2, 21.5±0.2, 22.4±0.2, 22.9±0.2, 23.1±0.2, 26.3±0.2, 27.1±0.2, 27.8±0.2, 28.5±0.2, 30.7±0.2, and 31.56±0.2 °2θ.

28. The Form III of N,N-dimethyltryptamine of claims 20, characterized an XRPD pattern substantially similar to that shown in FIG. 2.

29. canceled

30. The Form III of N,N-dimethyltryptamine of claims 20, which exhibits a Differential Scanning Calorimetry (DSC) thermogram comprising an endothermic peak at 44±5° C. when heated at a rate of 10° C./minute.

31. The Form III of N,N-dimethyltryptamine of claim 20, which exhibits substantially no weight loss at a temperature under 200±5° C. as measured by thermogravimetric (TGA) analysis.

32. The Form III of N,N-dimethyltryptamine ef-any-of claims 20-31, having a melting point of 39±5° C.

33. A pharmaceutical composition comprising Form Ill or Form IV of N,N-dimethyltryptamine and a pharmaceutically acceptable excipient.

34. The pharmaceutical composition of claim 33, comprising Form III of N.N-dimethyltryptamine.

35. canceled

36. canceled

37. The pharmaceutical composition of claim 33, comprising Form IV of N.N-dimethyltryptaminefurther.

38. canceled

39. A method of treating post-traumatic stress disorder (PTSD), depression, or an anxiety disorder in a subject in need thereof, comprising administering a therapeutically effective amount of a Form IV of N,N-dimethyltryptamine, a Form III of N,N-dimethyltryptamine, or a pharmaceutical composition of a Form Ill or a Form IV of N,N-dimethyltryptamine to the subject.

40. canceled

41. canceled

42. canceled
